(19) United States
(12) Patent Application Publication
(10) Pub. No.: US 2014/0163310 A1
(43) Pub. Date: Jun. 12, 2014

(54) Vibrating Baby Attachment

(71) Applicant: Alanna Innes, Embro (CA)

(72) Inventor: Alanna Innes, Embro (CA)

(21) Appl. No.: 14/085,373

(22) Filed: Nov. 20, 2013

Related U.S. Application Data
(60) Provisional application No. 61/736,256, filed on Dec. 12, 2012.

(51) Int. Cl. A61M 21/02

(52) U.S. Cl. CPC A61M 21/02
USPC 600/27

(57) ABSTRACT

A vibrating baby attachment device is disclosed which is a portable apparatus that connects to baby gear and equipment and which vibrates to calm a baby. The vibrating baby attachment device comprises a base component that houses a motor assembly and a power component. The power component is in communication with the motor assembly for supplying power to the motor assembly. The base component further comprises a plurality of switches for controlling the motor assembly and power component. The base component can also comprise a strap and/or a clip to secure the device to the baby equipment, and padding to provide a buffer against unwanted sounds. Further, the device can comprise an audible component housed within the base component, which acts to produce music and white noise, and is typically electrically powered.

### CROSS-REFERENCE

This application claims priority from Provisional Patent Application Ser. No. 61/736,256 filed Dec. 12, 2012.

### BACKGROUND

Babies often become anxious or stressed in situations in which a caregiver cannot hold them or give them proper attention, such as during a car ride. Further, many babies cry or otherwise act out if they are stationary in a stroller or crib for an extended period of time. Vibrations can be very soothing for infants, but there is not always an accessible way to provide this therapeutic solution in a safe, consistent and effective manner. An effective solution necessary.

The present invention converts any baby seat or other baby equipment into a soothing, vibrating device, and keeps an infant happy and calm in scenarios that may otherwise be stressful. The vibrating baby attachment device plays music or white noise to further relax a baby, and eliminates the need to buy a variety of different musical or vibrating contraptions for an infant. The device provides a range of uses and can be secured to almost any baby equipment, such as cribs, strollers, car seats, bassinets, swings, and anywhere else a baby may need to be soothed, and the device transports easily for use on the go.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one aspect thereof, comprises a vibrating baby attachment device which is a portable apparatus that connects to pre-existing baby gear and equipment and which vibrates to calm a baby. The vibrating baby attachment device comprises a base component that houses a motor assembly and a power component. The power component is in communication with the motor assembly for supplying power to the motor assembly.

The base component further comprises a plurality of switches for controlling the motor assembly and power component. For example, the base component can comprise a power switch for turning on and off the device, a switch or a dial that controls strength of vibrations from the device, and a volume switch or dial for controlling the volume of the device. The base component can also comprise a strap and/or a clip to secure the device to the baby gear or equipment.

In a preferred embodiment, the base component comprises padding or other suitable cushioning material secured to the back surface of the base component to provide a buffer against unwanted sounds. Further, the vibrating baby attachment device comprises an audible component housed within the base component, which acts to produce music and white noise, and is typically electrically powered.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

FIG. 1A illustrates a front perspective view of the vibrating baby attachment device in accordance with the disclosed architecture.

FIG. 1B illustrates a rear perspective view of the vibrating baby attachment device in accordance with the disclosed architecture.

FIG. 1C illustrates a perspective view of the inner mechanism of the vibrating baby attachment device in accordance with the disclosed architecture.

FIG. 2 illustrates a perspective view of the vibrating baby attachment device in use with a car seat in accordance with the disclosed architecture.

FIG. 3 illustrates a perspective view of the vibrating baby attachment device in use with a baby swing in accordance with the disclosed architecture.

FIG. 4 illustrates a perspective view of the vibrating baby attachment device in use with a stroller in accordance with the disclosed architecture.

FIG. 5 illustrates a perspective view of the vibrating baby attachment device in use with a bassinet in accordance with the disclosed architecture.

### DESCRIPTION OF PREFERRED EMBODIMENTS

The innovation is now described with reference to the drawings, wherein like reference numerals are used to refer to like elements throughout. In the following description, for purposes of explanation, numerous specific details are set forth in order to provide a thorough understanding thereof It may be evident, however, that the innovation can be practiced without these specific details. In other instances, well-known structures and devices are shown in block diagram form in order to facilitate a description thereof.

The present invention converts any baby seat or other baby equipment into a soothing, vibrating device, and keeps an infant happy and calm in scenarios that may otherwise be stressful. The vibrating baby attachment device plays music or white noise to further relax a baby, and eliminates the need to buy a variety of different musical or vibrating contraptions for an infant. The device provides a range of uses on existing items including cribs, strollers, car seats, bassinets, swings, and anywhere else a baby may need to be soothed, and the device transports easily for use on the go.

The disclosed vibrating baby attachment device comprises a base component that houses a motor that spins a weight causing vibrations and a power component. The power component is in communication with the motor assembly for supplying power to the motor assembly. The base component further comprises a plurality of switches for controlling the motor assembly and power component. The base component can also comprise a strap and/or a clip to secure the device to the baby equipment, and padding to provide a buffer against unwanted sounds from friction or rubbing against whatever the device is attached to. Further, the device can comprise an audible component housed within the base component, which acts to produce music and/or white noise, and is typically electrically powered.

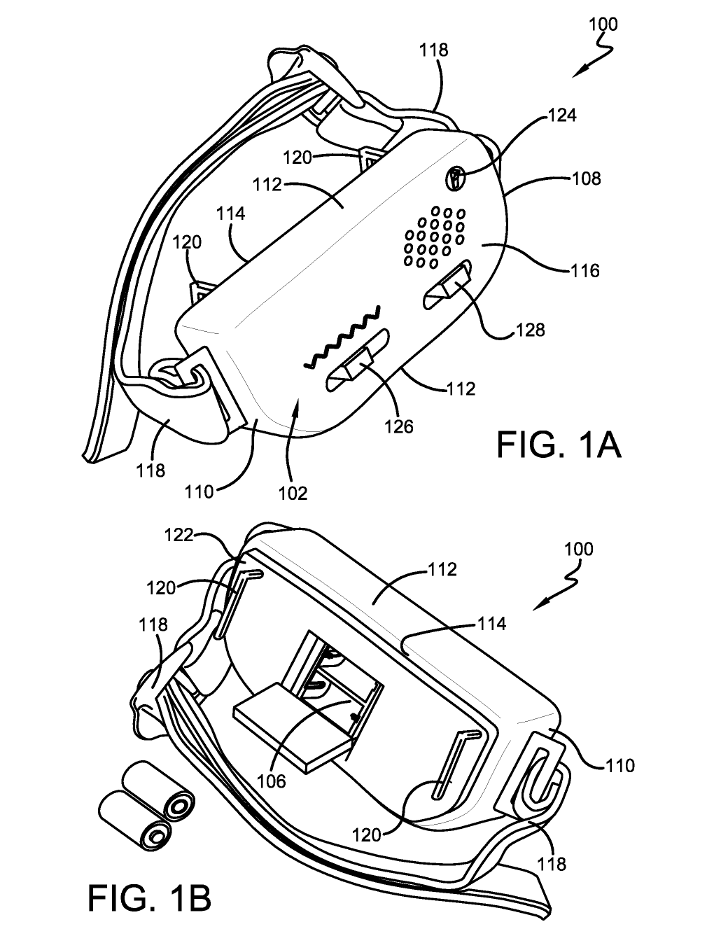

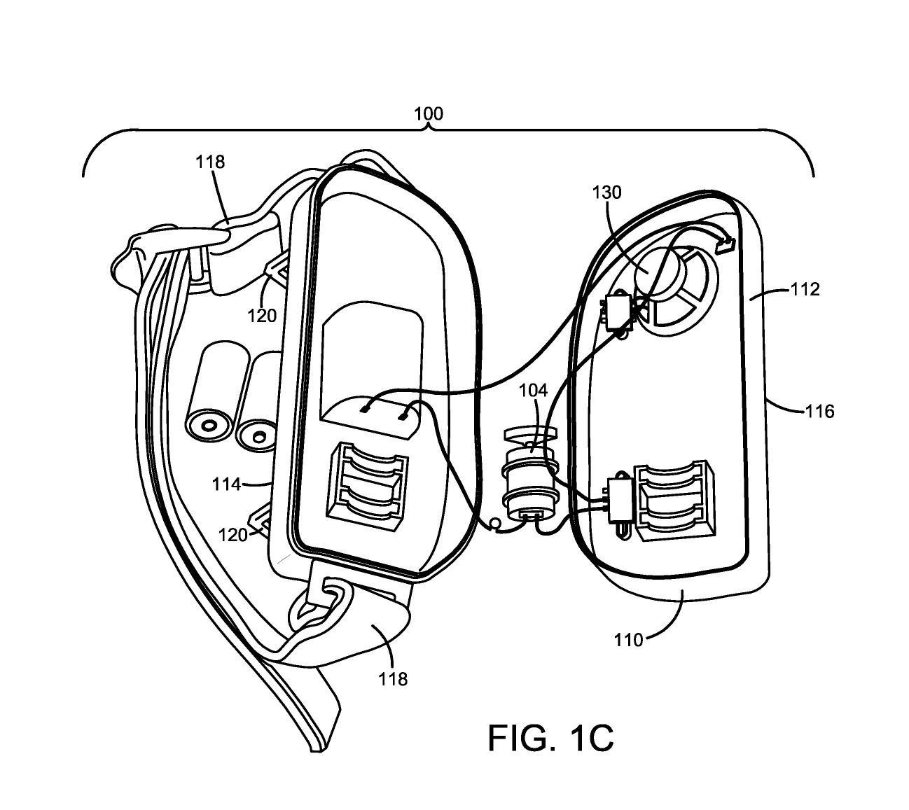

Referring initially to the drawings, FIGS. 1A-1C illustrate the vibrating baby attachment device 100 which is a portable apparatus that may be attached to baby gear and/or equipment to impart a vibrating motion thereto to calm a baby. The vibrating baby attachment device 100 comprises a base component 102, a motor assembly 104 housed within the base component 102; and a power component 106 in communication with the motor assembly 104 for supplying power to the motor assembly 104 (as shown in FIG. 1B).

The base component 102 comprises a first end 108, a second end 110, opposing sides 112, a back surface 114 and a front surface 116 (as shown in FIG. 1A). Typically, the base component 102 can be any suitable shape as is known in the art without affecting the overall concept of the invention, such as rectangular, square, circular, oval, triangular, etc. The base component 102 would generally be constructed of homogeneous plastics, such as polyvinyl chloride (PVC), acrylonitrile butadiene styrene (ABS), polycarbonate (PC), etc., composite polymers, or recycled material, though any other suitable material may be used to manufacture the base component 102 as is known in the art without affecting the overall concept of the invention.

The base component 102 can also comprise a variety of colors and designs to suit user and manufacturing preference. While the shape and size of the base component 102 may vary greatly depending on the wants and needs of a user, the base component 102 is approximately between 4 and 5 inches long as measured from a first end 108 to a second end 110, and approximately between 2 and 3 inches wide as measured from opposing sides 112, and approximately between 1 and 1.5 inches thick as measured from a front surface 116 to a back surface 114.

The base component 102 further comprises a strap 118 and/or a clip 120 to secure the device 100 to the baby gear or equipment. A strap 118 can be secured to each side 112 of the base component 102 or to any suitable position on the base component 102 such as the back surface 114, etc. The strap 118 would be secured via any suitable securing means as is known in the art, such as gluing, fasteners, welding, etc. The straps 118 would further comprise hook and loop fasteners or other securing means to secure (tighten) the straps 118 around objects of varying size. The clip 120 can also be secured to the base component 102 at any suitable position, such as the back surface 114 of the base component 102. The clip 120 would be secured via any suitable securing means as is known in the art, such as gluing, fasteners, welding, etc. The clip 120 secures the base component 102 to an object during use.

Further, the base component 102 can comprise padding 122 secured to the back surface 114 or any other suitable position on the base component 102. The padding 122 can be any suitable cushioning material as is known in the art to provide a buffer against unwanted sounds.

The vibrating baby attachment device 100 further comprises a motor assembly 104 housed within the base component 102 (as shown in FIG. 1C). The motor assembly 104 is typically secured within a compartment within the base component 102, but the motor assembly 104 can be secured in any suitable position on the exterior surface or within the base component 102 as is known in the art without affecting the overall concept of the invention. The motor assembly 104 functions as a prior art motor as is known in the art, and acts to spin a weight which vibrates the base component 102, which in turn vibrates whatever the base component 102 is secured to, such as a bassinet, a baby stroller, a baby car seat, a baby swing, etc.

Furthermore, the vibrating baby attachment device 100 comprises a power component 106 housed within the base component 102 and in communication with the motor assembly 104 for supplying power to the motor assembly 104. The power component 106 is typically secured within a compartment within the base component 102, but the power component 106 can be secured in any suitable position on the exterior surface or within the base component 102 as is known in the art without affecting the overall concept of the invention. The power component 106 functions to provide power to the motor assembly 104 and is in communication with the motor assembly 104. Typically, the power component 106 is a battery, but can be any suitable power source as is known in the art, such as an electrical power cord that is plugged into an electrical receptacle to provide power to the motor assembly 104, or a corded car adapter.

The vibrating baby attachment device 100 further comprises an audible component 130 housed within the base component 102. The audible component 130 is typically secured within a compartment within the base component 102, but the audible component 130 can be secured in any suitable position on the exterior surface or within the base component 102 as is known in the art without affecting the overall concept of the invention. The audible component 130 functions as a prior art audible component as is known in the art, and acts to produce music and/or white noise, and is typically electrically powered.

The base component 102 of the vibrating baby attachment device 100 further comprises a plurality of switches, dials or other controls. More specifically, the base component 102 may comprise a power switch 124 for turning on and off the device 100, a switch 126 that controls strength of vibrations from the device 100, and a volume switch 128 for controlling the volume of audible component 130. However, the power switch could be encompassed within the switch 126 that controls the strength of vibrations from the device 100, such that the switch 126 controls both vibrations and power. For example, if a user would move the switch 126 to the left, the device 100 would be turned off, and if the switch 126 is moved once to the right, then the device 100 would be turned on, and if the switch 126 is moved farther to the right, the strength of the vibrations could be adjusted. Thus, one button or switch would control both power and strength of vibrations so as not to complicate the device 100. Additionally, the volume switch 128 could also have two positions to control two levels of volume, one for the music volume and one for the white noise volume.

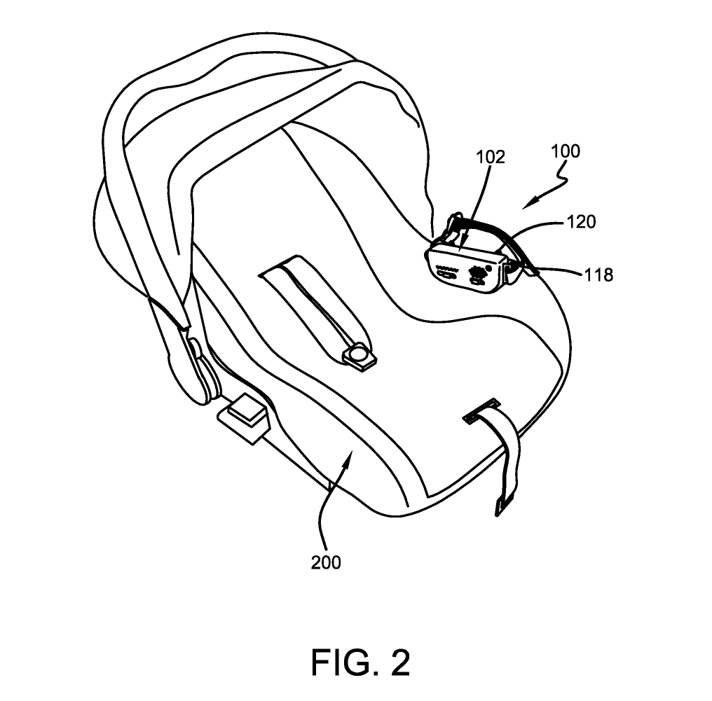

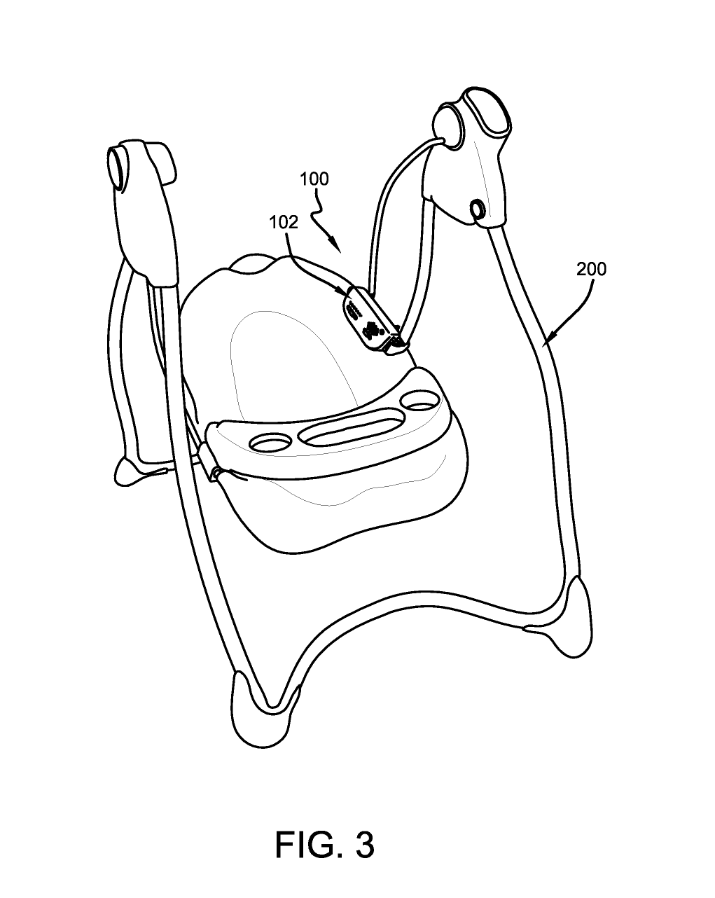

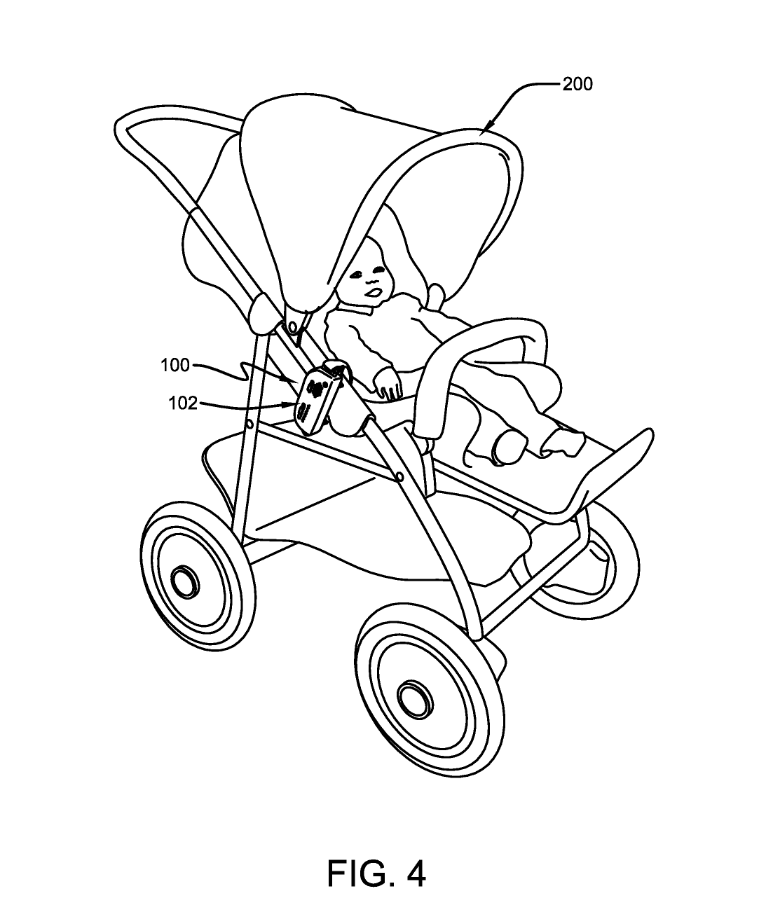

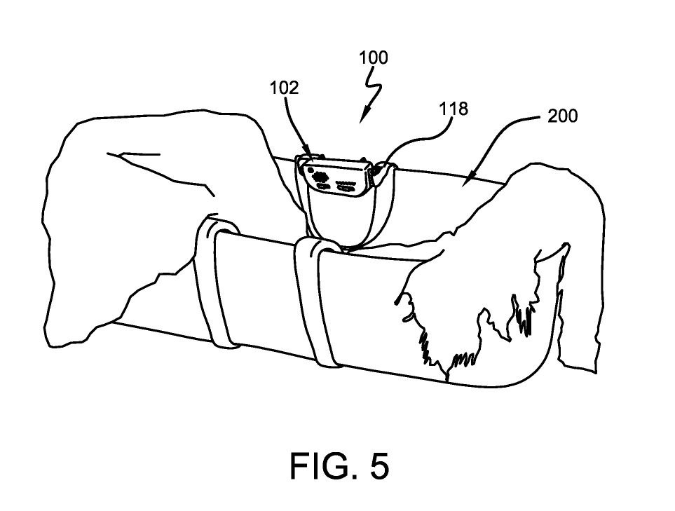

FIGS. 2-5 illustrate the vibrating baby attachment device 100 in use with various baby equipment 200. As stated supra, the vibrating baby attachment device 100 comprises a base component 102 that houses a motor assembly 104 and a power component 106. The power component 106 is in communication with the motor assembly 104 for supplying power to the motor assembly 104.

The base component 102 further comprises a plurality of switches 124, 126, 128 for controlling the motor assembly 104 and power component 106. For example, the base component 102 can comprise a power switch 124 for turning on and off the device 100, a switch 126 that controls strength of vibrations from the device 100, and a volume switch 128 for controlling the volume of audible component 130. The base component 102 can also comprise a strap 118 and/or a clip 120 to secure the device 100 to the baby gear or equipment, further the vibrating baby attachment device 100 can also be built into the baby gear or equipment at the point of manufacture, but can also be removable as well.

Furthermore, the base component 102 can comprise padding 122 or other suitable cushioning material secured to the back surface 114 of the base component 102 to provide a buffer against unwanted sounds. Further, the vibrating baby attachment device 100 comprises an audible component 130 housed within the base component 102, which acts to produce music and/or white noise, and is typically electrically powered.

In operation, a user (not shown) secures the vibrating baby attachment device 100 to a predetermined piece of baby equipment, via the straps 118 and/or the clip 120. The user then turns the power switch 124 on and adjusts the volume switch 128 and/or the switch 126 that controls the strength of vibrations from the device to the desired settings. The device 100 then provides a soothing vibration which keeps an infant happy and calm in scenarios that may otherwise be stressful. The vibrating baby attachment device 100 can also play music and/or white noise to further relax a baby, as needed.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A vibrating baby attachment device comprising:
a base component comprising a first end, a second end, opposing sides, a back surface and a front surface;
a motor assembly housed within the base component; and
a power component housed within the base component and in communication with the motor assembly for supplying power to the motor assembly.

2. The vibrating baby attachment device of claim 1, wherein the power component is a battery and the base component comprises a compartment for encasing the battery.

3. The vibrating baby attachment device of claim 1, wherein the power component is a power cord that is plugged into an electrical receptacle or a car adapter to provide power to the motor assembly.

4. The vibrating baby attachment device of claim 1, further comprising an audible component housed within the base component.

5. The vibrating baby attachment device of claim 4, wherein the audible component produces sounds.

6. The vibrating baby attachment device of claim 1, further comprising a power switch secured to the base component for turning on and off the device.

7. The vibrating baby attachment device of claim 6, further comprising a switch secured to the base component for controlling strength of vibrations from the device.

8. The vibrating baby attachment device of claim 1, further comprising a strap secured to each of the opposing sides of the base component.

9. The vibrating baby attachment device of claim 1, further comprising a clip secured to the back surface of the base component.

10. The vibrating baby attachment device of claim 1, further comprising padding secured to the back surface of the base component for buffering unwanted sounds.

11. A vibrating baby attachment device comprising:
a base component comprising a first end, a second end, opposing sides, a back surface and a front surface;
a motor assembly housed within the base component;
an audible component housed within the base component for producing sounds; and
a battery housed within a compartment of the base component and in communication with the motor assembly for supplying power to the motor assembly and the audible component.

12. The vibrating baby attachment device of claim 11, further comprising a power switch secured to the base component for turning on and off the device.

13. The vibrating baby attachment device of claim 12, further comprising a switch secured to the base component for controlling strength of vibrations from the device.

14. The vibrating baby attachment device of claim 13, further comprising a volume switch secured to the base component for adjusting the volume of the sounds.

15. The vibrating baby attachment device of claim 11, further comprising padding secured to the back surface of the base component for buffering unwanted sounds.

16. The vibrating baby attachment device of claim 11, further comprising a strap secured to each of the opposing sides of the base component.

17. The vibrating baby attachment device of claim 11, further comprising a clip secured to the back surface of the base component.

18. A vibrating baby attachment device comprising:
a base component comprising a first end, a second end, opposing sides, a back surface and a front surface;
a motor assembly housed within the base component for producing a vibrational movement;
an audible component housed within the base component for producing sounds;
a battery housed within a compartment of the base component and in communication with the motor assembly for supplying power to the motor assembly and the audible component; and
a clip and a strap secured to the back surface of the base component for securing the device to an object.

19. The vibrating baby attachment device of claim 18, further comprising padding secured to the back surface of the base component for buffering unwanted sounds.

20. The vibrating baby attachment device of claim 18, further comprising a power switch, a switch for controlling strength of vibrations, and a volume switch secured to the base component for adjusting the volume of the sounds.
